(19) United States
(12) Patent Application Publication
(10) Pub. No.: US 2012/0190884 A1
(43) Pub. Date: Jul. 26, 2012

(54) METHOD FOR PREPARING DIFLUOROACETIC ACID ESTERS

(75) Inventor: Olivier Buisine, Saint Genis Laval (FR)

(73) Assignee: RHODIA OPERATIONS, Aubervilliers (FR)

(21) Appl. No.: 13/388,831

(22) PCT Filed: Jul. 29, 2010
(86) PCT No.: PCT/EP2010/061064

§ 371 Date: Apr. 16, 2012

(30) Foreign Application Priority Data
Aug. 3, 2009 (FR) 09/03826

(51) Int. Cl. C07C 67/14

(52) U.S. Cl. 560/227

(57) ABSTRACT

A method for preparing difluoroacetic acid esters is described. The method can include reacting difluoroacetyl fluorine with an aliphatic or cycloaliphatic alcohol in the presence of a heterogeneous mineral base.

The present invention relates to a method for preparing difluoroacetic acid esters.

The invention relates to the preparation of alkyl and cycloalkyl esters of difluoroacetic acid.

The invention relates more particularly to the preparation of methyl or ethyl difluoroacetate.

The difluoroacetic acid esters are known products described in the literature.

One route of access consists of reacting an alcohol R₁OH with a difluoroacetyl fluoride of formula H—CF₂—COF. We may refer in particular to the preparation described in EP-A 0 694 523.

The alcoholysis reaction is conducted continuously, in the absence of catalyst. However, when the reaction is carried out according to a batch mode, the presence of a reaction catalyst is required in order to have a reaction time compatible with industrial application.

The method described in EP-A 0 694 523 recommends the use of a tertiary amine, and more particularly triethylamine, as catalyst.

The recourse to said catalyst, which is an organic base, involves extra cost due to the presence of triethylamine and the need to recover it at the end of the reaction, so that it can be recycled.

Another drawback of this method is that it leads to wastes that are very polluting owing to the presence of amines.

The applicant proposes a method that is able to overcome the aforementioned drawbacks.

A method has now been found, which constitutes the object of the present invention, for preparing an ester of difluoroacetic acid, characterized in that it comprises the reaction of difluoroacetyl fluoride with an aliphatic or cycloaliphatic alcohol, in the presence of a heterogeneous mineral base.

According to the method of the invention, an aliphatic or cycloaliphatic alcohol, designated hereinafter “alcohol”, is reacted with difluoroacetyl fluoride, in the presence of a heterogeneous mineral base.

The method of the invention involves an alcohol, which can be represented by the following formula:

R₁—OH (I)

in said formula, R₁ represents a substituted or unsubstituted hydrocarbon group, which can be an alkyl or cycloalkyl group.

In the context of the invention, “alkyl” means a linear or branched hydrocarbon chain having from 1 to 15 carbon atoms and preferably from 1 or 2 to 10 carbon atoms.

Examples of preferred alkyl groups are notably methyl, ethyl, propyl, isopropyl, butyl, isobutyl, t-butyl.

“Cycloalkyl” means a cyclic or monocyclic hydrocarbon group comprising from 3 to 8 carbon atoms, preferably a cyclopentyl or cyclohexyl group.

It should be noted that in these groups, one or more hydrogen atoms can be replaced with a substituent (for example, halogen), provided it does not interfere with obtaining the desired product.

In particular, the hydrocarbon chain can preferably bear one or more fluorine atoms.

Thus, R₁ can represent a fluorinated or perfluorinated alkyl group comprising from 1 to 10 carbon atoms and from 1 to 21 fluorine atoms, preferably from 3 to 21 fluorine atoms.

The alcohols preferably used in the method of the invention correspond to formula (I), in which R₁ represents an alkyl group having from 1 to 4 carbon atoms.

As examples of alcohols corresponding to formula (I), we may more preferably mention methanol, ethanol, isopropanol, 2,2,2-trifluoroethanol, 2,2-difluoroethanol, 1,1-difluoroethanol, pentafluoroethanol, hexafluoroisopropanol and cyclohexanol.

Regarding difluoroacetyl fluoride, it can be available alone or mixed with hydrofluoric acid: the amount of hydrofluoric acid present can be such that the ratio of the number of moles of hydrofluoric acid to the number of moles of difluoroacetyl fluoride can reach a value equal to 10, and is preferably between 1 and 7.

According to the method of the invention, the reaction between difluoroacetyl fluoride and the alcohol is carried out in the presence of a base.

The ratio of the number of moles of alcohol to the number of moles of difluoroacetyl fluoride can vary between 0.8 and 2, and is preferably between 0.95 and 1.05.

The method of the invention involves a base, whose function is to trap the hydrofluoric acid formed by the reaction.

The base used is a heterogeneous mineral base.

“Mineral base” means, in the present text, a mineral salt having at least a pKₐ above 8, preferably between 8 and 14.

The pKₐ is defined as the ionic dissociation constant of the acid/base pair when water is used as solvent. It is defined at 20° C.

“Heterogeneous” means that the mineral salt is insoluble in the reaction mixture.

It is possible to use a salt of a monovalent metal and/or of a divalent metal, preferably an alkali metal and/or alkaline-earth metal.

As more specific examples of salts, we may mention the carbonates, hydrogen carbonates, phosphates, hydrogen phosphates of an alkali metal, preferably sodium, potassium or cesium; of an alkaline-earth metal preferably magnesium, calcium, barium; of a group IIB metal, preferably zinc.

In the present text, reference will be made hereinafter to the periodic table of the elements published in the Bulletin de la Société Chimique de France, No. 1 (1966).

Among the bases, sodium carbonate or potassium carbonate is preferably selected.

The metal hydroxides are bases that are to be excluded for application of the method of the invention.

According to one feature of the method of the invention, the base is used in solid form, generally in the form of a powder and more particularly in a ground form in order to have a finer granulometry.

The operation of grinding of the base can be carried out in any type of grinding mill that is resistant to corrosion of the base (for example made of stainless steel).

The amount of base employed is such that the ratio of the number of moles of base to the number of moles of difluoroacetyl fluoride is preferably between 0.5 and 3, and more preferably between 1 and 2.

The reaction can be carried out in the presence or in the absence of an organic solvent.

A solvent that is inert in the reaction conditions is selected.

In the choice of solvent, a solvent that dissolves the product obtained is preferred.

Suitable solvents are nonpolar organic solvents such as aromatic hydrocarbons, aliphatic, cycloaliphatic or aromatic hydrocarbons, halogenated or not, or more polar organic solvents such as notably ether-oxides, nitriles.

As nonlimiting examples of such solvents, we may mention aromatic hydrocarbons such as notably benzene, toluene, xylenes, ethylbenzene, diethylbenzenes, trimethylbenzenes, petroleum cuts consisting of a mixture of alkylbenzenes and notably the cuts of the Solvesso® type.

Halogenated aliphatic or aromatic hydrocarbons can also be used, and we may mention: perchlorinated hydrocarbons such as notably tetrachloroethylene; partially chlorinated hydrocarbons such as dichloromethane, trichloromethane, dichloroethane, tetrachloroethane, trichloroethylene, 1-chlorobutane, 1,2-dichlorobutane; monochlorobenzene, dichlorobenzenes or mixtures thereof; trifluoromethylbenzene, trifluoromethoxybenzene.

Ethers can also be used as solvents. We may mention for example aliphatic, cycloaliphatic or aromatic ether-oxides, and more particularly methyl-tert-butyl ether, dipentyl oxide, diisopentyl oxide, ethylene glycol dimethyl ether (or 1,2-dimethoxyethane), diethylene glycol dimethyl ether (or 1,5-dimethoxy-3-oxapentane), anisole, veratrole or cyclic ethers, for example dioxane, tetrahydrofuran.

A solvent of the nitrile type can also be selected. We may notably mention the aliphatic or aromatic nitriles, preferably acetonitrile, propionitrile, butanenitrile, isobutanenitrile, pentanenitrile, 2-methylglutaronitrile, adiponitrile, benzonitrile, tolunitrile, malonitrile, 1,4-benzonitrile.

A mixture of organic solvents can also be used.

It may be advantageous to add an organic solvent in order to facilitate stirring of the medium.

The amount of organic solvent used is preferably selected in such a way that the concentration by weight of difluoroacetyl fluoride in the solvent is between 20 and 100 wt. %, preferably between 20 and 80 wt. %. The reaction is generally carried out at a temperature between 0° C. and 10° C. when it is carried out under atmospheric pressure.

The temperature can be selected between 0° C. and 100° C., preferably between 0° C. and 40° C., under autogenous pressure of the reactants.

The alcoholysis reaction is generally carried out preferably under controlled atmosphere of inert gases. It is possible to establish an atmosphere of rare gases, preferably argon, but it is more economical to use nitrogen.

The method of the invention is simple to carry out.

The reactants can be introduced according to many variants, but certain are preferred.

A preferred embodiment consists of preparing a basis consisting of alcohol, optionally the organic solvent and the mineral base and then gradually introducing, preferably by bubbling, the difluoroacetyl fluoride.

After stirring the reaction mixture at the selected temperature, at the end of the reaction we obtain an ester of difluoroacetic acid corresponding to the following formula:

H—CF₂—COOR₁ (II)

in this formula, R₁ has the meaning given above.

The product obtained is recovered by conventional means.

Generally, the salts formed are separated first, according to the techniques of solid/liquid separation, preferably by filtration.

The salts can in particular be the excess of mineral base and the salts formed by the reaction, most often a fluoride of sodium or of potassium.

The ester formed can be recovered from the liquid phase, separated by conventional means and more particularly by distillation.

The method of the invention is advantageously carried out in equipment that is resistant to corrosion by the reaction mixture.

For this purpose, corrosion-resistant materials are selected for the portion in contact with the reaction mixture such as alloys based on molybdenum, chromium, cobalt, iron, copper, manganese, titanium, zirconium, aluminum, carbon and tungsten sold under the HASTELLOY® trademarks or alloys of nickel, chromium, iron, manganese with addition of copper and/or molybdenum marketed under the designation INCONEL® and more particularly the alloys HASTELLOY C 276 or INCONEL 600, 625 or 718.

Stainless steels can also be selected, such as austenitic steels [Robert H. Perry et al., Perry's Chemical Engineers' Handbook, Sixth Edition (1984), page 23-44], and more particularly the stainless steels 304, 304 L, 316 or 316 L. A steel is used having a nickel content of max. 22 wt. %, preferably between 6 and 20%, and more preferably between 8 and 14%.

Steels 304 and 304 L have a nickel content between and 12% and steels 316 and 316 L have a nickel content between 10 and 14%.

Steels 316 L are more particularly selected.

It is also possible to use equipment consisting of or coated with a polymer compound resistant to corrosion by the reaction mixture. We may notably mention materials such as PTFE (polytetrafluoroethylene or Teflon) or PFA (perfluoroalkyl resins), high-density polyethylene. Use of an equivalent material remains within the scope of the invention.

As other materials that may be suitable for being in contact with the reaction mixture, we may also mention derivatives of graphite.

The method of the invention is particularly advantageous for application in batch mode.

The method of the invention employs an inexpensive base mineral, which does not require recovery for recycling.

Moreover, it does not lead to amine-containing effluents.

Examples of carrying out the invention are given below. These examples are given for purposes of illustration and are nonlimiting.

The degree of conversion and the yield obtained are determined in the examples.

The degree of conversion (TT) corresponds to the ratio of the number of moles of difluoroacetyl fluoride transformed to the number of moles of difluoroacetyl fluoride used.

The yield (RR) corresponds to the ratio of the number of moles of ester of difluoroacetic acid formed to the number of moles of difluoroacetyl fluoride used.

The analyses are carried out by ¹H-NMR and ¹⁹F-NMR.

### EXAMPLE 1

An autoclave made of Teflon®, equipped with a gas inlet and with a stirring system of the Rushton turbine type, is charged with ethanol (50 g; 1.09 mol) and sodium carbonate (116.2 g; 1.1 mol).

The autoclave is closed and the temperature of the mixture is lowered to 5° C. A gas stream of difluoroacetyl fluoride (mass flow rate of 190 g/h) is then introduced into the mixture for 30 minutes, keeping the temperature below 15° C.

The amount of difluoroacetyl fluoride added is 95 g (0.97 mol).

It is stirred for one hour at a temperature of 15° C., then the top of the reactor is purged with nitrogen for 30 minutes, allowing the temperature to return to room temperature (20° C.). Then the autoclave is degassed.

The reaction mixture is then filtered, and the salts are rinsed with 100 mL of dichloromethane.

The filtrates are combined and distilled at atmospheric pressure.

The fraction collected has a boiling point between 96 and 99° C.

92 g of ethyl difluoroacetate is obtained.

The yield is 77 wt. %.

### EXAMPLE 2

An autoclave as described in example 1 is charged with methanol (80 g; 2.5 mol) and sodium carbonate (270.3 g; 2.55 mol).

The autoclave is closed and the temperature of the mixture is lowered to 5° C.

A gas stream of difluoroacetyl fluoride (mass flow rate of 190 g/h) is then added to the mixture for 94 minutes, keeping the temperature below 15° C.

The amount of difluoroacetyl fluoride added is 256 g (2.5 mol).

It is stirred for one hour at a temperature of 15° C., then the top of the reactor is purged with nitrogen for 30 minutes, allowing the temperature to return to room temperature (20° C.). Then the autoclave is degassed.

The reaction mixture is then filtered, and the salts are rinsed with 250 mL of dichloromethane.

The filtrates are combined and distilled at atmospheric pressure.

The fraction collected has a boiling point between 85 and 86° C.

189 g of methyl difluoroacetate is obtained.

The yield is 69 wt. %.

### EXAMPLE 3

An autoclave as described in example 1 is charged with ethanol (50 g; 1.09 mol), xylene (800 mL) and sodium carbonate (477 g; 4.5 mol).

The autoclave is closed and the temperature of the mixture is lowered to 5° C.

A mixture of difluoroacetyl fluoride and anhydrous hydrofluoric acid (molar ratio HF/difluoroacetyl fluoride=1/3.2) is then added to the mixture so as to keep the temperature below 10° C.

The amount of difluoroacetyl fluoride added is 105 g (1.07 mol) and the amount of HF added is 68.6 g (3.4 mol).

It is stirred for one hour at a temperature of 15° C., then the top of the reactor is purged with nitrogen for 30 minutes, allowing the temperature to return to room temperature (20° C.). Then the autoclave is degassed.

The reaction mixture is distilled at atmospheric pressure.

The fraction collected has a boiling point between 96 and 98° C.

87.8 g of ethyl difluoroacetate is obtained.

The yield is 65 wt. %.

1. A method for preparing an ester of difluoroacetic acid, the method comprising reacting difluoroacetyl fluoride with an aliphatic or cycloaliphatic alcohol, in the presence of a heterogeneous mineral base.

2. The method as claimed in claim 1, wherein the alcohol corresponds to the following formula:
R₁—OH (I)
wherein in said formula, R₁ represents a substituted or unsubstituted hydrocarbon group, which can be an alkyl or cycloalkyl group.

3. The method as claimed in claim 2, wherein the group R₁ represents an alkyl group having from 1 to 4 carbon atoms.

4. The method as claimed in claim 2, wherein the group R₁ represents a fluorinated or perfluorinated alkyl group comprising from 1 to 10 carbon atoms and from 1 to 21 fluorine atoms.

5. The method as claimed in claim 1, wherein the alcohol is selected from the group consisting of: methanol, ethanol, isopropanol, 2,2,2-trifluoroethanol, 2,2-difluoroethanol, 1,1-difluoroethanol, pentafluoroethanol, hexafluoroisopropanol and cyclohexanol.

6. The method as claimed in claim 1, wherein the difluoroacetyl fluoride is mixed with hydrofluoric acid.

7. The method as claimed in claim 1, wherein the ratio of the number of moles of alcohol to the number of moles of difluoroacetyl fluoride ranges from 0.8 to 2.

8. The method as claimed in claim 1, wherein the base is a salt of a monovalent metal and/or of a divalent metal.

9. The method as claimed in claim 8, wherein the salt is selected from the group consisting of: a carbonate, a hydrogen carbonate, a phosphate, a hydrogen phosphate of an alkali metal; of an alkaline-earth metal; or a group IIB metal.

10. The method as claimed in claim 9, wherein the salt is sodium carbonate or potassium carbonate.

11. The method as claimed in claim 8, wherein the base is used in a solid form.

12. The method as claimed in claim 8, wherein the amount of base used is such that the ratio of the number of moles of base to the number of moles of difluoroacetyl fluoride ranges from 0.5 to 3.

13. The method as claimed in claim 1, wherein the reaction is carried out in the presence of an organic solvent.

14. The method as claimed in claim 13, wherein the organic solvent is selected from the group consisting of: an aromatic hydrocarbon, a halogenated aliphatic, cycloaliphatic or aromatic hydrocarbon; an ether-oxide and a nitrile.

15. The method as claimed in claim 13, wherein the amount of organic solvent is such that the concentration by weight of difluoroacetyl fluoride in the solvent ranges from 20 wt. % to 100 wt. %.

16. The method as claimed in claim 1, wherein the reaction is carried out at a temperature between 0° C. and 10° C. when it is carried out under atmospheric pressure.

17. The method as claimed in claim 1, wherein the reaction is carried out at a temperature between 0° C. and 100° C., under autogenous pressure of the reactants.

18. The method as claimed in claim 1, wherein the alcoholysis reaction is carried out under controlled atmosphere of an inert gas.

19. The method as claimed in claim 1, wherein the ester obtained is methyl or ethyl difluoroacetate.

20. The method as claimed in claim 4, wherein the group R₁ comprises 3 to 21 fluorine atoms.

21. The method as claimed in claim 7, wherein the ratio of the number of moles of alcohol to the number of moles of difluoroacetyl fluoride ranges from 0.95 to 1.05.

22. The method as claimed in claim 8, wherein the base is a salt of an alkali metal and/or alkaline-earth metal.

23. The method as claimed in claim 9, wherein the alkali metal is sodium, potassium or cesium.

24. The method as claimed in claim 9, wherein the alkaline-earth metal is magnesium, calcium or barium.

25. The method as claimed in claim 9, wherein the Group II B metal is zinc.

26. The method as claimed in claim 11, wherein the solid form is a powder or a ground form.

27. The method as claimed in claim 12, wherein the ratio of the number of moles of base to the number of moles of difluoroacetyl fluoride ranges from 1 to 2.

28. The method as claimed in claim 12, wherein the concentration by weight of difluoroacetyl fluoride in the solvent ranges from 20 wt. % to 80%.

29. The method as claimed in claim 17, wherein the temperature ranges from 0° C. to 40° C.

30. The method as claimed in claim 18, wherein the inert gas is nitrogen.
